(19) United States
(12) Patent Application Publication
(10) Pub. No.: US 2024/0173495 A1
(43) Pub. Date: May 30, 2024

(54) VENTILATION ASSISTANCE

(71) Applicants: THE UNIVERSITY OF SYDNEY, Sydney (AU); WESTERN SYDNEY LOCAL HEALTH DISTRICT, Westmead (AU)

(72) Inventors: Matthew CROTT, Killarney Heights (AU); Mark TRACY, Glenbrook (AU); Murray HINDER, Leura (AU); Matthew BOUSTRED, Mosman (AU)

(73) Assignees: THE UNIVERSITY OF SYDNEY, Sydney, NS (AU); WESTERN SYDNEY LOCAL HEALTH DISTRICT, Westmead, NS (AU)

(21) Appl. No.: 18/551,022

(22) PCT Filed: Mar. 25, 2022
(86) PCT No.: PCT/AU2022/050272

§ 371 Date: Sep. 18, 2023

(30) Foreign Application Priority Data
Mar. 29, 2021 (AU) 2021900924

(51) Int. Cl. A61M 16/00; A61B 5/00; A61B 5/087

(52) U.S. Cl. CPC A61M 16/0003; A61B 5/087; A61B 5/742; A61M 2016/003; A61M 2205/15; A61M 2205/3334; A61M 2205/502

(57) ABSTRACT

A ventilation assistance device and a method of using a ventilation assistance device for providing ventilation to a subject. The ventilation assistance device comprises a duct configured to be connectable between a source of gas and an interface device, for establishing fluid communication between the source of gas and the interface device for delivering gas to a subject's airway; a flow sensor configured to determine a flow volume of gas in the duct; and an operator interface configured to represent a measure of gas received by the subject by way of a plurality of indicators, each indicator being representative of the measure being within a particular range suitable for respective reference subjects having respective characteristics; wherein the operator interface is configured to display one of the plurality of indicators based on the flow volume determined by the flow sensor to represent to an operator the characteristic of the reference subject for which the gas received is suitable.

### CROSS-REFERENCE TO RELATED APPLICATION

This application claims the benefit of priority of Australian Provisional Patent Application No. 2021900924 filed on Mar. 29, 2021, the content of which is incorporated herein by reference in its entirety for all purposes.

### FIELD OF THE INVENTION

The present invention relates to the field of ventilation assistance and in particular to a ventilation assistance device and a method of operating such a ventilation assistance device. In particular, such ventilation assistance device may be used for resuscitation and resuscitation training, including resuscitation of infants and in training operators in infant resuscitation.

### BACKGROUND OF THE INVENTION

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

Methods of ventilation assistance, including for resuscitating infants, are known. These methods typically involve an operator operating a resuscitation device to deliver gas such as air and/or oxygen to the airway of the infant. Self-inflating bags and ventilation machines are examples of known resuscitation devices.

Self-inflating bag resuscitators (‘bag-mask devices’) require an operator to manually compress a self-inflating bag to deliver a volume of gas to the airway of the infant. After the volume of gas has been delivered to the infant, the operator allows the self-inflating bag to self-inflate by releasing pressure on the self-inflating bag. Subsequently, the operator can compress the self-inflating bag again to deliver another volume of gas to the airway of the infant.

Ventilation machines are machines that are able to cyclically deliver a volume of gas to the airway of the infant. Ventilation machines typically provide several controls that an operator can adjust to control one or more characteristics of the volume of gas delivered to the airway of the infant.

For infant resuscitation, babies come in different shapes and sizes. As a result they each have different requirements. Particularly for manual ventilation, but also relevant for machine-assisted ventilation, one of the most important and most variable needs is the amount of gas being delivered to the baby's lungs. A small baby needs a small amount of air and a large baby needs a larger amount of air. Too much gas and the baby's lungs can be damaged through overdistension, or oxygen flow restricted to the brain, leading to serious complications such as brain or lung damage. Conversely, too little gas, resulting in a lack of oxygen for the infant, has the potential to lead to brain damage or even death. This concept of ensuring the volume of air is correct for the size of the infant is often not taught (or not taught sufficiently clearly) during newborn resuscitation training, where trainees are generally trained just to observe the baby's chest rising and falling with ventilation. While respiratory function monitors are able to monitor airflow and display information on tidal volumes in millilitres, airway pressures or other unit, operators without advanced training are unable to correctly interpret such readouts, particularly in resuscitation situations, and such errors in interpretation could lead to disastrous consequences. This precludes the use of respiratory function monitors in neonatal resuscitation by operators without advanced training.

Embodiments of the present invention seek to address at least one of the above problems.

### SUMMARY OF THE INVENTION

According to a first aspect, the present invention provides a ventilation assistance device comprising:
a duct configured to be connectable between a source of gas and an interface device, for establishing fluid communication between the source of gas and the interface device for delivering gas to a subject's airway;
a flow sensor configured to determine a flow volume of gas in the duct; and
an operator interface configured to represent a measure of gas received by the subject by way of a plurality of indicators, each indicator being representative of the measure being within a particular range suitable for respective reference subjects having respective characteristics;
wherein the operator interface is configured to display one of the plurality of indicators based on the flow volume determined by the flow sensor to represent to an operator the characteristic of the reference subject for which the gas received is suitable.

According to a second aspect, the present invention provides a method of using a ventilation assistance device for providing ventilation to a subject, the method comprising the steps of:
using a duct of the ventilation assistance device for establishing fluid communication between a source of gas and an interface device for delivering gas to the subject's airway;
using a flow sensor of the ventilation assistance device to determine a flow volume of gas in the duct;
using an operator interface of the ventilation assistance device to represent a measure of gas received by the subject by way of a plurality of indicators, each indicator being representative of the measure being within a particular range suitable for respective reference subjects having respective characteristics, wherein the operator interface is configured to display one of the indicators based on the flow volume determined by the flow sensor to represent to an operator the characteristic of the reference subject for which the gas received is suitable; and
the operator maintaining a current operation mode of performing the ventilation assistance if the characteristic of the reference subject for which the gas received is suitable matches the subject or adjusting the current operation mode of performing the ventilation assistance if otherwise.

### BRIEF DESCRIPTION OF THE DRAWINGS

Preferred embodiments of the present invention will be described, by way of examples only, with reference to the accompanying figures, wherein:

FIG. 1 shows a ventilation assistance device according to an embodiment of the present invention;

FIG. 2(A) shows a sample display of a ventilation assistance device according to an example embodiment;

FIG. 2(B) shows a sample display of a ventilation assistance device according to an example embodiment;

FIG. 2(C) shows a sample display of a ventilation assistance device according to an example embodiment;

FIG. 2(D) shows a sample display of a ventilation assistance device according to an example embodiment;

FIG. 2(E) shows a sample display of a ventilation assistance device according to an example embodiment;

FIG. 2(F) shows a sample display of a ventilation assistance device according to an example embodiment;

FIG. 3 shows a patient ventilation set-up incorporating the ventilation assistance device of FIG. 1;

FIG. 4 shows a schematic diagram illustrating a ventilation assistance device according to an example embodiment; and

FIG. 5 shows a flowchart illustrating a method for ventilation assistance according to an example embodiment.

### DETAILED DESCRIPTION OF THE EMBODIMENTS

An example embodiment of the present invention provides the user with quantitative data relating to ventilation assistance that is easy to interpret and intuitive to use. A ‘symbolic logic’ of the feedback-based solution according to an example embodiment of the invention allows users to adjust, optimize and improve their technique during actual ventilation assistance and during training, so that they are optimally equipped for action during manual ventilation, in particular in babies following birth.

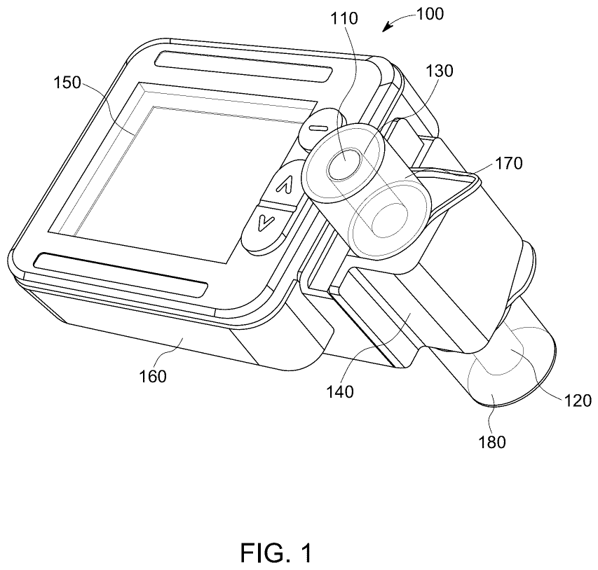

FIG. 1 shows a ventilation assistance device 100 according to an example embodiment of the present invention. The ventilation assistance device 100 has a duct in the form of a tube 130 with a first opening 110 at a first end, a second opening 120 at a second end, as well as a flow sensor 140 and associated control circuitry inside the housing 160 of the ventilation assistance device 100, and an operator interface in the form of a display 150 and associated control circuitry inside the housing 160 of the ventilation assistance device 100. It will be appreciated that the example shape and configuration of the ventilation assistance device and its components are not limited to the example shape and configuration as shown for the example embodiment in FIG. 1. It will further be appreciated that the circuitry for the display 150 and the flow sensor 140 may be separately implemented, or may be implemented in one processing circuit. It will further be appreciated that the circuitry may be integrally provided in the housing 160 circuit and/or may be externally provided with suitable interconnection media.

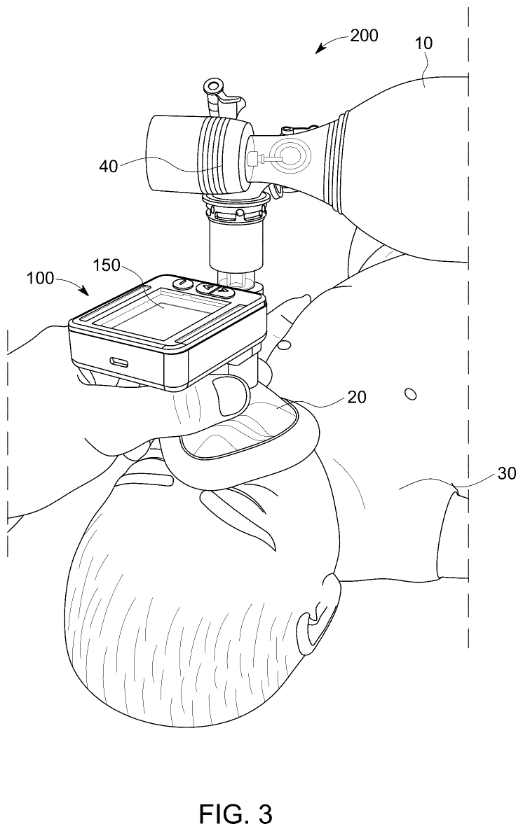

The first opening 110 is configured to be coupled in fluid communication with a source of gas (generally, air and/or oxygen) 10 (see FIG. 3). In FIG. 3, the gas source 10 is illustrated as a self-inflating bag, however, the gas source 10 may be a ventilator, anaesthetic bag. T-piece resuscitator, or any other suitable device as understood in the art for supplying gas for use in resuscitation or continuous positive airway pressure (CPAP) support. The source of gas 10 will be referred to below as “gas source 10” for convenience. With reference again to FIG. 1, the first opening 110 may be coupled to an outlet of the gas source 10 by a friction fit, respective complementary threads, a Luer fitting, or any other suitable means capable of coupling the first opening 110 in fluid communication with the gas source 10. In the embodiment shown in FIG. 1, an outer tube portion 170 is provided around the first opening 110 for friction fit with a tube-shaped coupler on the gas source 10.

The second opening 120 is configured to be coupled in fluid communication with an interface device 20 (see FIG. 3). The interface device 20 may be a face mask, endotracheal tube, laryngeal mask airway, nasal cannulae, or any other suitable device as understood in the art that is capable of delivering gas to the airway of a subject 30. The interface device will be referred to below as “interface device 20” for convenience. The second opening 120 may be coupled to the interface device 20 by any suitable means, as discussed above with reference to the coupling between first opening 110 and gas source 10, applied to the second opening 120 and/or the second end 125. In the embodiment shown in FIG. 1, an outer tube portion 180 is provided around the first opening 120 for friction fit with a tube-shaped coupler on the interface device 20.

Referring still to FIG. 1, the tube 130 extends between the first opening 110 and the second opening 120 to provide a fluid connection therebetween. In other words, when the first opening 110 is coupled in fluid communication with a gas source 10 and the second opening 120 is coupled in fluid communication with an interface device 20, and the gas source 10 and the interface device 20 are in fluid communication with each other via the tube 130.

The flow sensor 140 is configured to measure the rate of a gas flow through the tube 130. The flow sensor 140 may be a single sensor configured to measure flow through the tube 130 from the second opening 120 to the first opening 110 and to measure flow through the tube 130 from the first opening 110 to the second opening 120, i.e. bi-directionally. Alternatively, the flow sensor 140 may comprise two uni-directional flow sensors, one configured to measure flow through the tube 130 from the first opening 110 to the second opening 120 and another flow sensor to measure flow through the tube 130 from the second opening 120 to the first opening 110. The flow measured may be sensed as a flow rate (e.g. in L/min) or as a flow volume (e.g. mL supplied or returned in a single ventilatory cycle), preferably for a single ventilation cycle.

The flow sensor 140 may be any suitable single sensor or two sensors as understood in the art capable of measuring flow through the tube 130. This includes through a hot-wire anemometer, differential pressure pneumotach, micro-electromechanical systems (MEMS) sensors, such as thermal MEMS sensors or MEMS piezoelectric sensors, or any other suitable flow sensing method as understood in the art. Depending on the type of sensor(s) used, the flow sensor 140 may be positioned within the tube 130 (e.g. hot-wire anemometers and MEMS flow sensors) or external to the tube 130 (e.g. ultrasonic flow meters) inside the housing 160. In the example embodiment, the flow sensor comprises a thermal MEMS sensor positioned internal to the tube 130.

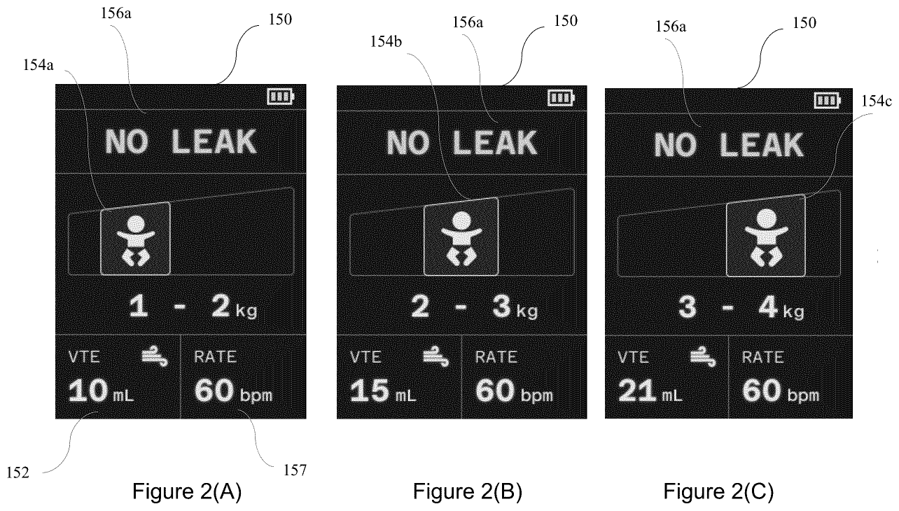

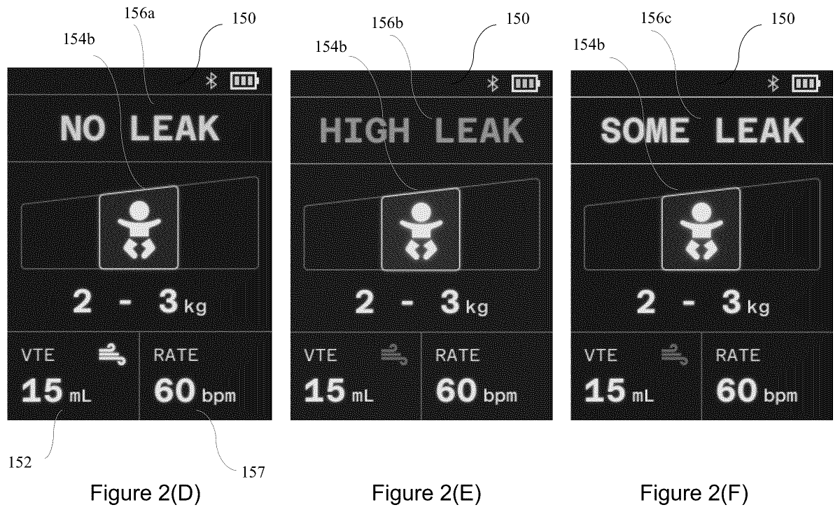

FIGS. 2(A) to (F) show example screen shots of the display 150 according to an example embodiment of the invention. The display 150 has a ventilatory volume indicator 152, infant size indicators 154, leakage indicators 156, and ventilatory rate indicator 157. The ventilatory volume indicator 152, infant size indicators 154, and leakage indicators 156a-c represent characteristics of the flow of gas through the tube 130 for each ventilation cycle, as will be discussed in detail below.

In use, the first opening 110 and the second opening 120 of the ventilation assistance device 100 are coupled in fluid communication with the gas source 10 and a interface device 20, respectively, and the interface device 20 is positioned to deliver gas to the airway of a subject 30. For example, if the interface device 20 is a resuscitation mask (see FIG. 3), the resuscitation mask is placed over the nose and mouth of the subject 30. Together, the air source 10, the ventilation assistance device 100, and the interface device 20 may form a patient ventilation set-up 200 (see FIG. 3).

With reference now to FIG. 3, the patient ventilation set-up 200 is being used to cyclically supply gas to the subject 30 and expel air subsequently exhaled by the subject 30. Each cycle of supplying gas to the subject 30 and expelling air subsequently exhaled by the subject 30 is referred to herein as a ventilation cycle. Accordingly, each ventilation cycle comprises supplying gas to the subject 30, which is at least partially inhaled by the subject 30, and expelling at least some of the gas subsequently exhaled by the subject 30.

For each ventilation cycle, an operator operates the gas source 10 to supply gas (i.e. a inspiratory volume) to the subject 30 and expel at least some of a volume of gas that is subsequently exhaled by the subject 30 (i.e. the expiratory volume). For each ventilation cycle, the inspiratory volume flows from the air source 10, through the tube 130 of the ventilation assistance device 100, to the interface device 20, where the inspiratory volume is at least partially inhaled by the subject 30. At least some of the inhaled gas that is subsequently exhaled by the subject 30 (i.e. the expiratory volume) then flows into the interface device 20, through the tube 130 of the ventilation assistance device 100, and is expelled to atmosphere (e.g. via a one-way valve 40 in the gas source 10).

As an example, the gas source 10 may be a manual resuscitator comprising a self-inflating bag as shown in FIG. 3 (a ‘bag-valve mask device’). The operator operates such a device by compressing the self-inflating bag to partially deflate it, causing an inspiratory volume to be delivered from the air source 10 to the airway of the subject 30 via the ventilation assistance device 100 and the interface device 20. The operator then releases the self-inflating bag to allow it to reinflate, before compressing it once again to deliver another inspiratory volume to the subject 30. The frequency of the ventilation cycle is dependent on how often an operator compresses the self-inflating bag in a set time period. The inspiratory volume supplied to the airway of the subject 30 for each ventilation cycle is dependent on the amount of compression the operator applies to the self-inflating bag.

As another example, if the air source 10 is a flow-driven resuscitation device (a ‘flow-driven device’), the operator may operate the flow-driven device by adjusting one or more device controls, including the peak inspiratory pressure (PIP) and the positive end expiratory pressure (PEEP). The operator can then deliver each inspiratory volume by occluding a one-way valve with their finger to redirect the air through the ventilation assistance device 100 and the interface device 20, and can vary the frequency of ventilatory cycles by varying the frequency of these occlusions.

For each ventilatory cycle, the ventilation assistance device 100 in this example embodiment uses the flow sensor 140 to determine the expiratory volume of gas and the inspiratory volume of gas that flow through the tube 130 subject 30. For example, and with reference again to FIGS. 2(A) to (F), the calculated expiratory volume is displayed at the ventilatory volume indicator 152 (e.g. in mL) on the display 150.

Referring still to FIGS. 2(A) to (F), the infant size indicators 154a-c in the example embodiment include three infant size indicators 154a-c representing images of differently sized infants. Each infant size indicator 154a-c represents an infant weight range (e.g. in kilograms). As an example, infant size indicator 154a may represent an infant weight range between 0.5 to 1.4 kg, infant size indicator 154b may represent an infant weight range between 1.5 to 3.4 kg, and infant size indicator 154c may represent an infant weight range between 3.5 to 5 kg. Although FIGS. 2(A) to (F) only illustrate three infant size indicators 154a-c, it will be appreciated that more generally two or more infant size indicators may be provided, each representing a predefined infant weight range. It will also be appreciated that indicators of infant gestational age could be substituted for indicators of infant weight range as another characteristic of the infant.

For each ventilation cycle, the ventilation assistance device 100 is configured to display or highlight on the display 150 one of the infant size indicators 154a-c to indicate an infant weight range that is suitable for the calculated expiratory volume. In the example of FIGS. 2(B) and (D) to (F), for a given ventilation cycle, the ventilatory volume indicator 152 indicates a calculated expiratory volume of 15 mL and the infant size indicator 154b is highlighted, indicating that this expiratory volume is suitable for mid-size infants (e.g. infants weighing between 2 to 3 kg). As an example, the ventilation assistance device 100 may display or highlight on the display 150 the:

Infant size indicator 154a for expiratory volumes between 5 mL to 12 mL for 1-2 kg infants;

Infant size indicator 154b for expiratory volumes between 12 mL to 18 mL for 2-3 kg infants; and

Infant size indicator 154c for expiratory volumes between 18 mL to 24 mL for 3-4 kg infants.

In the example of FIGS. 2(A) to (F), infant size indicator 154b is highlighted by showing an image representing the infant size, while the other infant size indicators 155a, c are blank. Alternatively, the display 150 may be configured to display on the display 150 images representing different infant sizes and highlight e.g. by changing from outline to solid filling, the one indicator 154a-c corresponding to the calculated expiratory volume. It is also envisaged that the infant size indicators 154a-c may be displayed or highlighted using any other suitable methods as understood in the art (e.g. visual, audible, and/or haptic indications).

Expiratory volume is related to the tidal volume of the infant, which varies depending on the infant's size or gestational age. Due to this association, a larger expiratory volume is generally appropriate for a larger infant size, and a smaller expiratory volume is generally appropriate for a smaller infant size. Inspiratory volume that is measured before the interface device 20 might not correspond to tidal volumes if there is a significant leak around the interface device 20 that results in some or all of the gas being vented to atmosphere rather than being delivered to the infant.

If the interface device 20 is not correctly positioned, a substantial amount of the inspiratory volume may not be inhaled by the subject 30 and may escape (i.e. leak) to atmosphere. This may result in less than the required volume of gas being inhaled by the subject 30 for each ventilation cycle. As an example, if the interface device 20 is a resuscitation mask and it is not correctly positioned over the nose and mouth of the subject 30, some of the inspiratory volume may escape between the interface between the resuscitation mask and the subject 30's face.

For each ventilation cycle, the ventilation assistance device 100 is configured to display or highlight on the display 150 one of the leakage indicators 156a-c to indicate the amount of leakage occurring during each ventilation cycle. Leakage is determined based on the inspiratory volume and expiratory volume. It is calculated as the difference between the inspiratory volume and expiratory volume as a percentage of the inspiratory volume. As an example, an inspiratory volume of 10 mL and expiratory volume of 5 mL would give a leak of 50%.

In the example of FIGS. 2(A) to (F), the ventilation assistance device 100 may be configured to display or highlight on the display 150 the:

leakage indicator 156b if the ventilation assistance device 100 calculates that more than 60% of the inspiratory volume has been leaked;

leakage indicator 156c if the ventilation assistance device 100 calculates that between 30-60% of the inspiratory volume has been leaked; and

leakage indicator 156a if the ventilation assistance device 100 calculates that between 0-30% of the inspiratory volume has been leaked.

The leakage indicators 156a-c may indicate the amount of leakage using visual indications. As an example, the leakage indicators 156a-c may use words and font color (based on traffic light), as shown in FIGS. 2(A)-(F):
leakage indicator 156b may display “high leak” in red font color;
leakage indicator 156c may display “some leak” in yellow/amber font color; and
leakage indicator 156a may display “no leak” in green font color.

Alternatively, other visual, audible, and/or haptic indications may be used, such as, but not limited to, a traffic light system based only on color.

Based on the leakage indicator 156a-c displayed on the display 150, an operator may be able to adjust the interface device 20 and/or any other aspect of the patient ventilation set-up 200 (see FIG. 3) to reduce the amount of leakage per ventilation cycle. Reducing the amount of leakage may increase the likelihood of the correct volume of gas being inhaled by the subject 30 during each ventilation cycle.

The display 150 may be configured to only display the leakage indicator 156a-c corresponding to the calculated leakage as shown in FIGS. 2(A) to (F). Alternatively, display 150 may be configured to highlight display all the leakage indicators 156a-c and to highlight the one corresponding to the calculated leakage, e.g. using a higher intensity and/or by changing from outline to solid filling. It is also envisaged that the leakage indicators 156a-c may be displayed or highlighted using any other suitable methods as understood in the art (e.g. visual, audible, and/or haptic indications). Although FIGS. 2(A) to (F) illustrates three leakage indicators 156a-c, it will be appreciated that more or less leakage indicators may be provided, each representing a predefined amount of leakage (e.g. in percentage of the inspiratory volume). In the example embodiment, three different leakage indicators 156a-c are provided, however the number can be different in different embodiments, including only one indicator for indicating when the leak is low.

Ventilatory rate is also calculated and displayed, see ventilatory rate indicator 157. It is determined by measuring the frequency of the ventilations by detecting the beginning of each inspiration, and then then displaying the rate per minute.

The “subject” discussed above may be an infant or a manikin of an infant, as shown in FIG. 3. Accordingly, the flow monitor 100 may be used to assist with resuscitating an infant weighing within a particular weight range and/or to train operators to resuscitate infants falling within different weight ranges. If the subject 30 is a manikin of an infant, the manikin may include a face and an airway, and be designed to provide observable chest movement to simulate lung inflation and deflation of an infant during resuscitation. Accordingly, the manikin may be used to replicate ventilation of an infant. There are different manikin sizes available in the market to represent different age/weight/size infants. The “matching” of the suitable size indicator according to example embodiments can either be done through the given specifications (e.g. the manufacturer specifies that this is a manikin that represents a 3 kg baby) or by simulation (e.g. the trainer says ‘pretend this is a 2 kg baby’, as non-limiting examples.

It is noted that the present invention is not limited to being applied to infants or manikins of infants, but can be applied generally to humans, including to other target groups, including to older pediatric patients and adults, or manikins thereof.

Use of the ventilation assistance device 100 to resuscitate an infant as a non-limiting example will now be described.

The first opening 110 and the second opening 120 of the ventilation assistance device 100 are coupled in fluid communication with a gas source 10 and a interface device 20, respectively, as described above. The ventilation assistance device 100, gas source 10, and interface device 20 together may form a patient ventilation set-up 200, as shown in FIG. 3.

Subsequently, an operator will position the interface device 20 to deliver gas to the airway of the infant. As an example, if the interface device 20 is a resuscitation mask, the interface device 20 is placed over the nose and mouth of the infant. As another example, if the interface device 20 is an endotracheal tube or a laryngeal mask airway, the interface device 20 is partially inserted into the airway of the infant.

The operator operates the gas source 10 as described above to supply a volume of gas (i.e. a inspiratory volume) to the infant and to expel at least some of the volume of gas subsequently exhaled by the infant (i.e. the expiratory volume) for each ventilation cycle.

With reference to FIGS. 2(A) to (F), for each ventilation cycle, the ventilation assistance device 100 in the example embodiment uses the flow sensor 140 to calculate the expiratory volume, inspiratory volume, leakage, and ventilatory rate, as described above. Based on the calculated expiratory volume and leakage, the ventilation assistance device 100 displays on the display 150 the infant size indicator 154a-c and the leakage indicator 156a-c corresponding to the calculated expiratory volume and leakage, respectively. The ventilation assistance device 100 may also display on the display 150 the calculated inspiratory volume at the ventilatory volume indicator 152, for example alternatingly with the expiratory, or based on user control of the display 150.

The infant the operator is resuscitating will have particular physiological characteristics (e.g. weight, size and gestational age). Infants of different weight/size and/or gestational age will require to receive different gas volumes to be delivered to their airway during resuscitation. Receiving a gas volume that is too large for a particular infant may result in damage to the infant's lungs or brain, and receiving a gas volume that is less than a required volume may cause brain or organ damage to the infant or result in the death of the infant through a lack of oxygen. Accordingly, it is desirable to maintain the gas volume received by the infant for each ventilation cycle within a particular range depending on the weight/size of the infant.

Based on the infant size indicator 154a-c displayed on the display 150, the operator is able to observe if the gas volume received by the infant for a ventilation cycle is appropriate for the infant they are resuscitating. If the infant size indicator 154a-c displayed on the display 150 for a ventilation cycle corresponds to the weight/size of the infant being resuscitated, the operator continues to operate the resuscitation device source 10 using the current mode of operation. As an example, if the gas source 10 is a self-inflating bag, the operator will continue compressing the self-inflating bag to a similar degree for the next ventilation cycle so that a similar gas volume is received by the infant. As another example, if the gas source 10 is a flow-driven device, the operator does not adjust the control settings of the flow-driven device so that the same gas volume is received by the infant for the next ventilation cycle.

If the infant size indicator 154a-c displayed on the display 150 does not correspond to the weight/size of the infant being resuscitated, the operator will adjust their mode of operating the gas source 10 so that the gas volume received by the infant for subsequent ventilation cycles is appropriate for the infant being resuscitated.

For example, if the infant size indicator 154a-c displayed on the display 150 indicates that the calculated inspiratory volume corresponds to an infant weight range that is less than the infant being resuscitated, the operator will adjust their mode of operating the gas source 10 to increase the inspiratory volume delivered to the infant for the next ventilation cycle. As an example, if the gas source 10 is a self-inflating bag, the operator will increase the degree of compression of the self-inflating bag for the next ventilation cycle to increase the inspiratory volume delivered to the infant. As another example, if the gas source 10 is a flow-driven device, the operator may adjust the peak inspiratory pressure of the flow-driven device to increase the inspiratory volume delivered to the infant for the next ventilation cycle.

As another example, if the infant size indicator 154a-c displayed on the display 150 indicates that the calculated inspiratory volume corresponds to an infant weight range that is greater than the weight range of the infant being resuscitated, the operator will adjust their mode of operating the gas source 10 to decrease the inspiratory volume delivered to the infant for the next ventilation cycle. As an example, if the gas source 10 is a self-inflating bag, the operator will decrease the degree of compression of the self-inflating bag for the next ventilation cycle to decrease the inspiratory volume delivered to the infant. As another example, if the gas source 10 is a flow-driven device, the operator may adjust one or more controls of the flow-driven device to decrease the inspiratory volume delivered to the infant for the next ventilation cycle.

Based on the leakage indicator 156a-c displayed on the display 150, the operator is able to observe how much of the inspiratory volume is being leaked to atmosphere for each ventilation cycle. If the leakage indicator 156b or 156c is displayed on the display 150, the operator may adjust the interface device 20 and/or any other aspect of the patient ventilation set-up 200 in order to reduce the amount of leakage for the next ventilation cycle. If the leakage indicator 156a is displayed on the display 150, the operator may continue with the current mode of operation with regard to leakage.

Accordingly, during resuscitation of an infant, an operator may be able to use the ventilation assistance device 100 to determine if the gas volume received by the infant for each ventilation cycle is appropriate for the weight/size of the infant. If the inspiratory volume is not appropriate for the weight/size of the infant being resuscitated, the operator will be able to adjust their mode of operating the gas source 10 so that the inspiratory volume delivered to the infant for the next berating cycle is more appropriate for the weight/size of the infant being resuscitated.

Further, during resuscitation of an infant, an operator may be able to use the ventilation assistance device 100 to determine the amount of leakage occurring during each ventilation cycle. If the amount of leakage is not within an acceptable range (e.g. within 0-30%), the operator may adjust the interface device 20 and/or any other aspect of the patient ventilation set-up 200 in order to reduce the amount of leakage for the next ventilation cycle.

Use of the ventilation assistance device 100 to train operators to resuscitate infants of different sizes as another non-limiting example will now be described.

The method of using the ventilation assistance device 100 to train operators to resuscitate infants is similar to the method described above for resuscitating infants, except that a manikin of an infant will be used to replicate ventilation of an infant. Accordingly, the interface device 20 will be positioned on the manikin instead of an infant. The manikin may be a manikin as described above or any other suitable manikin as understood in the art that is suitable for replicating ventilation of an infant.

During a resuscitation training exercise, an operator may desire to simulate resuscitating an infant having a particular weight/size. The operator operates the gas source 10 as described above to deliver an inspiratory volume to the manikin and to expel at least some of the inspiratory volume of gas subsequently exhaled by the manikin (i.e. the exhaled volume) for each ventilation cycle.

Similar to that described above, the operator will be able to observe if the gas volume received by the manikin is appropriate for the weight/size of the infant they simulating in the resuscitation training exercise. If the inspiratory volume is not appropriate for the weight/size of the infant they are simulating during the resuscitation exercise, the operator will adjust their mode of operating the gas source 10 as described above so that the inspiratory volume delivered to the manikin for the next ventilation cycle is more appropriate for the weight/size of the infant the operator is simulating in the resuscitation exercise.

As described above, the operator may also adjust the interface device 20 and/or any other aspect of the patient ventilation set-up 200 based on the leakage indicator 156a-c displayed on the display 150 during each ventilation cycle.

The operator can perform further resuscitation training exercises using the ventilation assistance device 100 to simulate resuscitating infants of different weights/sizes. The ventilation assistance device 100 may therefore be used to train operators to become more familiar with operating the gas source 10 for resuscitating infants of different weight/size. Accordingly, based on the resuscitation training exercises using the ventilation assistance device 100, an operator may be able to more effectively and consistently deliver the appropriate inspiratory volume for each ventilation cycle to infants of different weights/sizes.

As FIG. 3 illustrates, ventilation assistance device 100 is positioned between the operator and the subject 30 with display 150 facing upwards, so that ready observation of the display 150 and the content viewable thereon is possible without the operator having to turn attention away from the subject 30. This continuous observation of subject 30, display 150 and interface device 20 avoids as far as possibly any interruption in or distraction from optimum operation of gas source 10, while providing constant feedback to the operator with regard to maintenance of that optimum operation by way of activation of the different indicators. It is noted that in example embodiments in which, audible, and/or haptic indications may be used, the orientation of the display may be less critical.

Further, the ventilation assistance device 100 may be used to train operators to correctly position the interface device 20 on an infant before commencing resuscitation of the infant in order to reduce leakage and how to adjust the interface device 20 and/or any other aspect of the patient ventilation set-up 200 in order to reduce the amount of leakage during each ventilation cycle.

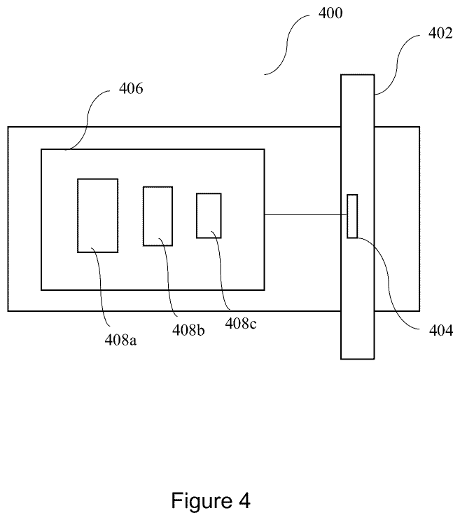

FIG. 4 shows a schematic drawing illustrating a ventilation assistance device 400 according to an example embodiment, comprising a duct 402 configured to be connectable between a source of gas and an interface device, for establishing fluid communication between the source of gas and the interface device for delivering gas to a subject's airway; a flow sensor 404 configured to determine a flow volume of gas in the duct 402; and an operator interface 406 configured to represent a measure of gas received by the subject by way of a plurality of indicators 408a-c, each indicator 408a-c being representative of the measure being within a particular range suitable for respective reference subjects having respective characteristics; wherein the operator interface 406 is configured to display one of the plurality of indicators 408a-c based on the flow volume determined by the flow sensor 404 to represent to an operator the characteristic of the reference subject for which the gas received is suitable.

The indicators 408a-c may be respective images representing an observable physical characteristic of the respective reference subjects.

The respective characteristics may relate to the size of the respective reference subjects.

The measure of the flow volume may be based on an expiratory volume flowing from the interface device through the duct as determined by the flow sensor 404 The measure of the flow volume may correspond to each expiratory volume determined by the flow sensor 404 from a continuous cycle of ventilation volumes.

The operator interface may be configured to provide a further representation of the flow volume, being a numerical representation.

The operator interface 406 may be configured to display a measure of leakage of gas from the interface device. The measure of leakage of gas may be determined based on an inspiratory volume determined by the flow sensor 404. The measure of the flow volume may correspond to each inspiratory volume determined by the flow sensor 404 from a continuous cycle of ventilation volumes.

The measure of leakage of gas may be determined based on a difference between the inspiratory volume determined by the flow sensor and the expiratory volume determined by the flow sensor 404. The measure of the flow volume may correspond to each inspiratory volume and each corresponding expiratory volume determined by the flow sensor 404 from a continuous cycle of ventilation volumes.

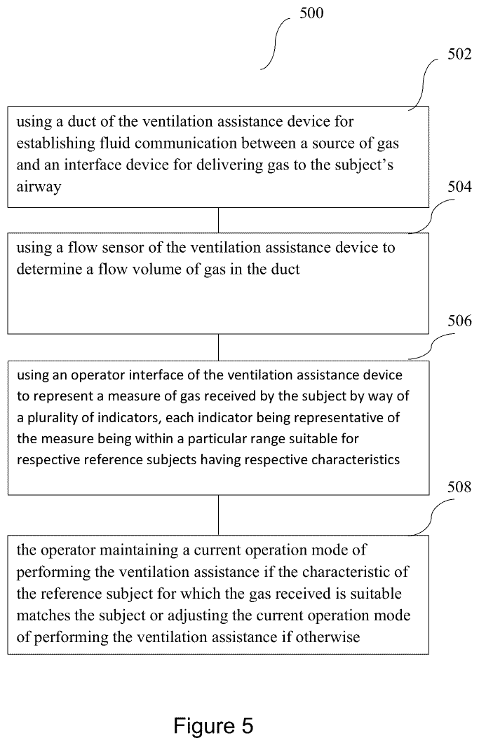

FIG. 5 shows a flowchart 500 illustrating a method of using a ventilation assistance device for providing ventilation to a subject, according to an example embodiment. At step 502. a duct of the ventilation assistance device is used for establishing fluid communication between a source of gas and an interface device for delivering gas to the subject's airway. At step 504. a flow sensor of the ventilation assistance device is used to determine a flow volume of gas in the duct. At step 506, an operator interface of the ventilation assistance device is used to represent a measure of gas received by the subject by way of a plurality of indicators, each indicator being representative of the measure being within a particular range suitable for respective reference subjects having respective characteristics, wherein the operator interface is used to display one of the indicators based on the flow volume determined by the flow sensor to represent to an operator the characteristic of the reference subject for which the gas received is suitable. At step 508, the operator maintains a current operation mode of performing the ventilation assistance if the characteristic of the reference subject for which the gas received is suitable matches the subject or adjusting the current operation mode of performing the ventilation assistance if otherwise.

The indicators may be respective images representing an observable physical characteristic of the respective reference subjects.

The respective characteristics may relate to the size of the respective reference subjects.

The measure of the flow volume may be based on an expiratory volume flowing from the interface device through the duct as determined by the flow sensor.

The measure of the flow volume may correspond to each expiratory volume determined by the flow sensor from a continuous cycle of ventilation volumes.

The method may comprise using the operator interface to provide a further representation of the flow volume, being a numerical representation.

The method may comprise using the operator interface to display a measure of leakage of gas from the interface device. The measure of leakage of gas may be determined based on an inspiratory volume determined by the flow sensor. The measure of the flow volume corresponds to each inspiratory volume may be determined by the flow sensor from a continuous cycle of ventilation volumes.

The measure of leakage of gas may be determined based on a difference between the inspiratory volume determined by the flow sensor and the expiratory volume determined by the flow sensor. The measure of the flow volume may correspond to each inspiratory volume and each corresponding expiratory volume determined by the flow sensor from a continuous cycle of ventilation volumes.

The method may comprise the operator maintaining a current operation mode of performing the ventilation assistance if the measure of leakage is indicated as acceptable; or the operator adjusting the current operation mode of performing the ventilation assistance if otherwise.

The subject may be a human or a manikin of a human.

Aspects of the systems and methods described herein, such as, but not limited to, the display and its control, the flow sensor and its control, and the measurement and processing of the flow data, may be implemented as functionality programmed into any of a variety of circuitry, including programmable logic devices (PLDs), such as field programmable gate arrays (FPGAs), programmable array logic (PAL) devices, electrically programmable logic and memory devices and standard cell-based devices, as well as application specific integrated circuits (ASICs). Some other possibilities for implementing aspects of the system include: microcontrollers with memory (such as electronically erasable programmable read only memory (EEPROM)), embedded microprocessors, firmware, software, etc. Furthermore, aspects of the system may be embodied in microprocessors having software-based circuit emulation, discrete logic (sequential and combinatorial), custom devices, fuzzy (neural) logic, quantum devices, and hybrids of any of the above device types. Of course the underlying device technologies may be provided in a variety of component types, e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, etc.

The various functions or processes disclosed herein may be described as data and/or instructions embodied in various computer-readable media, in terms of their behavioral, register transfer, logic component, transistor, layout geometries, and/or other characteristics. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. When received into any of a variety of circuitry (e.g. a computer), such data and/or instruction may be processed by a processing entity (e.g., one or more processors).

The above description of illustrated embodiments of the systems and methods is not intended to be exhaustive or to limit the systems and methods to the precise forms disclosed. While specific embodiments of, and examples for, the systems components and methods are described herein for illustrative purposes, various equivalent modifications are possible within the scope of the systems, components and methods, as those skilled in the relevant art will recognize. The teachings of the systems and methods provided herein can be applied to other processing systems and methods, not only for the systems and methods described above.

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive. Also, the invention includes any combination of features described for different embodiments, including in the summary section, even if the feature or combination of features is not explicitly specified in the claims or the detailed description of the present embodiments.

In general, in the following claims, the terms used should not be construed to limit the systems and methods to the specific embodiments disclosed in the specification and the claims, but should be construed to include all processing systems that operate under the claims. Accordingly, the systems and methods are not limited by the disclosure, but instead the scope of the systems and methods is to be determined entirely by the claims.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

1. A ventilation assistance device comprising:
a duct configured to be connectable between a source of gas and an interface device, for establishing fluid communication between the source of gas and the interface device for delivering gas to a subject's airway;
a flow sensor configured to calculate a volume of gas received by the subject based on determining a flow volume of gas in the duct; and
an operator interface configured to represent the calculated volume of gas received by the subject by way of a plurality of indicators, each indicator being representative of one of a plurality of reference subject groups having respective characteristics;
wherein the operator interface is configured to determine, based on the calculated volume of gas received by the subject, for which of the plurality of reference subject groups the calculated volume of gas received by the subject is suitable, and to display one of the plurality of indicators to represent to an operator the characteristic of the reference subject group for which the calculated volume of gas received by the subject has been determined to be suitable.

2. The device of claim 1, wherein the indicators are respective images representing an observable physical characteristic of the respective reference subject groups.

3. The device of claim 1, wherein the respective characteristics relate to the size of the respective reference subject groups.

4. The device of claim 1, wherein the calculated volume of gas received by the subject is based on an expiratory volume flowing from the interface device through the duct as determined by the flow sensor, and preferably wherein the calculated volume of gas received by the subject corresponds to each expiratory volume determined by the flow sensor from a continuous cycle of ventilation volumes.

5. (canceled)

6. The device of claim 1, wherein the operator interface is configured to provide a further representation of the calculated volume of gas received by the subject, being a numerical representation.

7. The device of claim 1, wherein the operator interface is configured to display a measure of leakage of gas from the interface device.

8. The device of claim 7, wherein the measure of leakage of gas is determined based on an inspiratory volume determined by the flow sensor.

9. The device of claim 8, wherein the measure of leakage of gas is based on each inspiratory volume determined by the flow sensor from a continuous cycle of ventilation volumes.

10. The device of claim 8, wherein the measure of leakage of gas is determined based on a difference between the inspiratory volume determined by the flow sensor and the expiratory volume determined by the flow sensor.

11. The device of claim 10, wherein the measure of leakage of gas is based on each inspiratory volume and each corresponding expiratory volume determined by the flow sensor from a continuous cycle of ventilation volumes.

12. A method of using a ventilation assistance device for providing ventilation to a subject, the method comprising the steps of:
using a duct of the ventilation assistance device for establishing fluid communication between a source of gas and an interface device for delivering gas to the subject's airway;
using a flow sensor of the ventilation assistance device to calculate a volume of gas received by the subject based on determining a flow volume of gas in the duct;
using an operator interface of the ventilation assistance device to represent the calculated volume of gas received by the subject by way of a plurality of indicators, each indicator being representative one of a plurality of reference subject groups having respective characteristics, wherein the operator interface is configured to determine, based on the calculated volume of gas received by the subject, for which one of the plurality of reference subject groups the calculated volume of gas received by the subject is suitable, and to display one of the plurality of indicators to represent to an operator the characteristic of the reference subject group for which the calculated volume of gas received by the subject has been determined to be suitable; and
the operator maintaining a current operation mode of performing the ventilation assistance if the characteristic of the reference subject group for which the calculated volume of gas received by the subject is suitable matches the subject or adjusting the current operation mode of performing the ventilation assistance if otherwise.

13. The method of claim 12, wherein the indicators are respective images representing an observable physical characteristic of the respective reference subject groups.

14. The method of claim 12, wherein the respective characteristics relate to the size of the respective reference subject groups.

15. The method of claim 12, wherein the calculated volume of gas received by the subject is based on an expiratory volume flowing from the interface device through the duct as determined by the flow sensor, and optionally wherein the measure of calculated volume of gas received by the subject corresponds to each expiratory volume determined by the flow sensor from a continuous cycle of ventilation volumes.

16. (canceled)

17. The method of claim 12, comprising using the operator interface to provide a further representation of the calculated volume of gas received by the subject, being a numerical representation.

18. The method of claim 12, comprising using the operator interface to display a measure of leakage of gas from the interface device.

19. The method of claim 18, comprising using the flow sensor to determine the measure of leakage of gas based on an inspiratory volume determined by the flow sensor.

20. The method of claim 19, wherein the leakage of gas is based on each inspiratory volume determined by the flow sensor from a continuous cycle of ventilation volumes.

21. The method of claim 19, wherein the measure of leakage of gas is determined based on a difference between the inspiratory volume determined by the flow sensor and the expiratory volume determined by the flow sensor, and optionally wherein the leakage of gas is based on each inspiratory volume and each corresponding expiratory volume determined by the flow sensor from a continuous cycle of ventilation volumes.

22. (canceled)

23. (canceled)

24. The method of claim 12, wherein the subject is a human or a manikin of a human.
